(19) United States
(12) Patent Application Publication
(10) Pub. No.: US 2021/0393572 A1
(43) Pub. Date: Dec. 23, 2021

(54) PHARMACEUTICALLY ACTIVE CANNABIS-BASED COMPOSITIONS AND METHODS OF USE FOR TREATING GASTROINTESTINAL CONDITIONS

(71) Applicant: VISCERAL THERAPEUTICS INC., Calgary (CA)

(72) Inventors: Jan KOWALCZEWSKI, Calgary (CA); Christopher ANDREWS, Calgary (CA)

(21) Appl. No.: 17/279,928

(22) PCT Filed: Sep. 23, 2019
(86) PCT No.: PCT/CA2019/051357

§ 371 Date: Mar. 25, 2021

Related U.S. Application Data
(60) Provisional application No. 62/738,091, filed on Sep. 28, 2018.

(51) Int. Cl. A61K 31/352; A61K 31/05; A61K 31/451; A61K 31/417; A61K 31/137; A61K 31/485; A61K 31/785; A61K 31/787; A61K 33/24

(52) U.S. Cl. CPC A61K 31/352; A61K 31/05; A61K 31/451; A61K 31/417; A61K 33/24; A61K 31/485; A61K 31/785; A61K 31/787; A61K 31/137

(57) ABSTRACT

According to embodiments herein, pharmaceutically active Cannabis-based compositions and methods of use for treating gastrointestinal conditions are provided. More specifically, a cannabis-based pharmaceutical composition and methods of use is provided, the composition comprising at least one cannabinoid and at least one motility agent. Without being limited by theory, the present compositions and methods of use may serve to either augment the positive or desired effects of the at least one cannabinoid constituent, and/or may serve to mitigate the negative or undesired effects of same, resulting in an improved Cannabis-based composition for treating, mitigating, or alleviating the symptoms of irritable bowel syndrome or irritable bowel syndrome-related disorders.

### RELATED APPLICATIONS

This application claims the benefit of U.S. Provisional Patent Application No. 62/738,091 entitled “Pharmaceutical Compositions and Methods of Use” filed Sep. 28, 2018, the entire contents of which are hereby incorporated by reference.

### FIELD

Pharmaceutically active cannabinoid compositions and methods of use for treating gastrointestinal conditions, and particularly for treating irritable bowel syndrome (IBS) and IBS-related symptoms, are provided.

### BACKGROUND

Irritable bowel syndrome (‘IBS’) is a chronic gastrointestinal disorder characterized by the presence of a group of symptoms relating to dysfunction of the lower bowel including compromised bowel function (e.g. diarrhea, constipation, etc.) and concomitant abdominal symptoms of pain and discomfort. IBS is the most common functional gastrointestinal (GI) disorder in North America, impacting between 10-25% of the population with a global pooled incidence of 11%.

Currently, IBS is diagnosed when a patient experiences persistent abdominal pain recurring, on average, at least one day per week for three consecutive months (i.e. within the three previous months), the pain being associated with two or more of the following criteria:
i. defecation;
ii. a change in the frequency of stool; and
iii. a change in form (appearance) of stool.

Accordingly, IBS can be subtyped into various categories including IBS-C, relating to IBS with predominant constipation, IBS-D, relating to IBS with predominant diarrhea, or IBS-M, relating to IBS with abnormal bowel movements that can include both constipation and diarrhea.

Pain is one of the most important concerns for the majority of individuals suffering from IBS, such pain affecting all aspects of quality of life, including the ability to work. It has been shown that the impact of IBS on work productivity is twice as great compared to the general population. Individuals suffering from IBS miss an average of approximately 13.4 days per year due to their symptoms, and approximately 34.4% of patients may have anxiety disorders, further impairing their quality of life and increasing their consumption of health care resources (e.g. up to 50% more than the general population). Unfortunately, research suggests that abdominal pain as the most difficult symptom to treat and the most necessary one to treat in order to improve quality of life.

There is currently no definite cure for IBS. The majority of treatments constitute managing patient discomfort and pain, however, an individual's response to treatment is highly dependent upon various disease triggers, including personal diet and stress. Moreover, it is commonly understood that individuals suffering from IBS have enteric nervous systems that are hypersensitive to normal stimuli, that is—triggers that would not normally affect others, such as food or mild stress, can provoke strong, painful responses in IBS sufferers. As a result, although a variety of therapies are used to treat the symptoms of IBS, few treatments are successful.

To date, according to the Canadian Association of Gastroenterology, the typical treatment for individuals suffering from IBS should comprise trying a diet consisting of low fermentable oligosaccharides, disaccharides, monosaccharides, polyols (FODMAP), where psyllium, but not wheat bran, supplementation is recommended. Alternative therapies, such as peppermint oil and probiotics are suggested, while herbal therapies and acupuncture are not. Cognitive behavioural therapy and hypnotherapy are suggested psychological therapies. Recommended pharmacological therapies include antispasmodics, certain antidepressants, eluxadoline, lubiprostone, and linaclotide, while motility agents, such as loperamide, cholestyramine and osmotic laxatives, are not recommended. Notwithstanding the foregoing, the nature of the IBS symptoms (diarrhea-predominant or constipation-predominant) should be considered as the choice of pharmacological treatments may vary. There is therefore a need for a novel and validated pharmaceutically effective composition for alleviating, mitigating, or treating individuals suffering from IBS or IBS-related symptoms.

The vast medicinal potential of plants in treating pain has been appreciated for generations. Cannabis, more commonly known as marijuana, is a genus of flowering plants that includes at least three species, Cannabis sativa, Cannabis indica, and Cannabis ruderalis. The therapeutic value of the active ingredients of Cannabis, termed cannabinoids, is well documented, particularly for the use in treating or improving chronic pain. The use of Cannabis, or a composition containing at least one cannabinoid, may prove beneficial in treating, mitigating, or alleviating the symptoms of IBS and IBS-related disorders.

Cannabinoids are a diverse class of chemical compounds that, inter alia, act on neuronal cannabinoid receptors. As a result, cannabinoids have an indirect anti-cholinergic effect mediated by the cannabinoid receptors, leading to the typical symptoms associated with cannabis consumption (e.g. dry mouth and eyes).

There are two main subtypes of the cannabinoid receptors in the central nervous system, cannabinoid-1 and cannabinoid-2. CB1 receptors are primarily located in the brain and have a consequential mediating effect on memory, cognition, and movement, while the CB2 receptors are primarily located in immune cells and play a role in immune mediation. Because cannabinoid receptors are the primary target of cannabinoids/cannabinoid agonists, Cannabis may offer a complimentary mode of action to supplement current IBS treatment. For example, in addition to pain relief, activation of cannabinoid receptors may modulate fecal urgency and pain in IBS patients. Cannabinoids may assist in reducing anxiety (e.g. via a modulatory role of the receptors on GABA and glutamate receptors across the amygdala and/or other forebrain areas). Moreover, cannabinoids may provide beneficial side effects to IBS sufferers including anti-spasmodic effects and the slowing of smooth muscle contractions and secretions in patients with IBS-D.

Research suggests, however, that long-term Cannabis use in the general population actually increases the risk for IBS and is correlated with increased cannabinoid hyperemesis syndrome, which is categorized by episodes of recurrent nausea, vomiting and crampy abdominal pain similar to IBS. Studies have also shown a positive association between anxiety disorder and Cannabis use. Symptoms often improve with the cessation of Cannabis and, as a result, Cannabis is not often prescribed for treating, mitigating, or alleviating the symptoms of IBS and IBS-related disorders. Indeed, many government agencies consider Cannabis to be a dangerous substance with no accepted usage for medical therapeutic purposes. A recent position statement of the Canadian Association of Gastroenterology for the use of Cannabis in GI disorders confirms that medical cannabinoid use should not replace Health Canada-approved medical therapy for treatment of any gastroenterologic or hepatologic disease if the approved therapy is available and has not been used.

Thus, there remains a need for a novel and validated pharmaceutically effective compositions for the treatment for individuals suffering from IBS. There is a particular need for well-defined compositions comprising at least one cannabinoid constituent that are effective in treating IBS and IBS-related symptoms.

### SUMMARY

According to embodiments herein, pharmaceutically active Cannabis-based compositions and methods of use for treating gastrointestinal conditions are provided. More specifically, a Cannabis-based pharmaceutical composition and methods of use is provided, the composition comprising at least one cannabinoid and at least one motility agent. Without being limited by theory, the present compositions and methods of use may serve to either augment the positive or desired effects of the at least one cannabinoid constituent, and/or may serve to mitigate the negative or undesired effects of same, resulting in an improved Cannabis-based composition for treating, mitigating, or alleviating the symptoms of irritable bowel syndrome or irritable bowel syndrome-related disorders.

In some embodiments, the present composition may comprise at least one cannabinoid constituent, the cannabinoid constituent comprising tetrahydrocannabinol (THC), cannabidiol (CBD), a phytocannabinoid, a terpenoid, and a combination thereof. The present composition may comprise a combination of THC and CBD.

In some embodiments, the at least one cannabinoid constituent may comprise at least one terpenoid, the at least one terpenoid selected from the group consisting of limonene, myrcene, α-pinene, linalool, β-caryophyllene, caryophyllene oxide, nerolidol and phytol, and a combination thereof.

In some embodiments, the present composition may also comprise at least one motility agent. The at least one motility agent may be at least one opioid receptor agonist, at least one opioid receptor antagonist, and a combination thereof.

In some embodiments, the at least one motility agent may comprise at least one opioid receptor agonist selected from the group consisting of a mu-, kappa-, and delta-opioid receptor agonist. In some embodiments, the at least one motility agent may be at least one opioid receptor agonist selected from the group consisting of meperidine, eluxadoline, loperamide, tapentadol. diphenoxylate, oxymorphindole, and a combination thereof.

In some embodiments, the at least one motility agent may comprise at least one opioid receptor antagonist selected from the group consisting of methylnaltrexone, naloxegol, naloxone, naldemedine.

In some embodiments, the present composition may also comprise at least one motility agent, the motility agent comprising a bile acid sequestrant. In some embodiments, the bile acid sequestrant may be selected from the group consisting of colestyramine and colestipol.

In some embodiments, the present composition may also comprise at least one motility agent, the motility agent comprising an anti-diarrheal agent. In some embodiments, the anti-diarrheal agent may be selected from the group consisting of bismuth subsalicylate, bismuth subgallate, attapulgite, crofelemer, and a combination thereof.

In some embodiments, the present composition may also comprise at least one motility agent, the motility agent comprising at least one enteric neuromodulator. In some embodiments, the at least one enteric neuromodulator is selected from the group consisting of atropine, hyoscine, scopolamine, dicyclomine, belladonna, trihexyphenidyl, benztropine, bethanechol, anisotropine, clinidium bromide, and combinations thereof.

In some embodiments, the present composition may also comprise at least one motility agent, the motility agent comprising at least one serotonin modulator. In some embodiments, the at least one serotonin modulator may be selected from the group consisting of prucalopride, renzapride, naronapride, cisapride, mosapride, tegaserod, trimebutine, ondansetron, granisetron, alosetron, and combinations thereof.

In some embodiments, the present composition may also comprise at least one motility agent, the motility agent comprising at least one gastrointestinal tract-specific calcium channel agonist. In some embodiments, the at least one gastrointestinal tract-specific calcium channel agonist may be selected from the group consisting of pinaverium, mebeverine, otilonium, and combinations thereof.

In some embodiments, the present composition may also comprise at least one motility agent, the motility agent comprising at least anti-foaming agent, defoaming agent, osmotic laxative, stimulant laxative, stool softeners, and combinations thereof. In some embodiments, the osmotic laxative, stimulant laxative, and stool softeners are selected from the group consisting of PEG 3350, milk of magnesia, lactulose, aloe vera, mineral oil, senna, cascara, docusate, and combinations thereof.

In some embodiments, the present composition may also comprise at least one motility agent, the motility agent comprising at least one soluble fiber, at least one insoluble fiber, and a combination thereof. In some embodiments, the at least one motility agent comprising at least one soluble fiber and at least one insoluble fiber may be selected from psyllium, inulin, guar gum, beta-glucans, pectins, resistant starches, and a combination thereof.

In some embodiments, the present composition may also comprise at least one motility agent, the motility agent comprising at least one microbiome modulator. In some embodiments, the at least one microbiome modulator may be selected from the group consisting of at least one probiotic, rifaximin, metronidazole, neomycin, a branched chain amino acid, ciprofloxacin, L-ornithine, L-aspartate, at least one prebiotic, or a combination thereof.

In some embodiments, the methods provided herein comprise administration of a composition as described herein. In some embodiments, the present methods may comprise formulating the compositions for administration to an individual suffering from irritable bowel syndrome or irritable bowel syndrome-related symptoms. In some embodiments, the present methods may comprise formulating the present compositions for administration to an individual for the treatment, mitigation, or alleviation of irritable bowel syndrome or irritable bowel syndrome-related symptoms. In some embodiments, the present methods may comprise formulating a pharmaceutically active amount of the composition for administration to an individual. In some embodiments, the present methods may comprise formulating the present compositions for use in the preparation of a medicament used to treat, mitigate, or alleviate the symptoms of irritable bowel syndrome or irritable bowel syndrome-related symptoms in an individual.

In some embodiments, methods are provided for treating, mitigating, or alleviating the symptoms of irritable bowel syndrome or irritable bowel syndrome-related symptoms. In some embodiments, the present methods comprise administering a pharmaceutically active amount of the present compositions to an individual suffering from irritable bowel syndrome or irritable bowel syndrome-related symptoms.

The following description describes various embodiments of the present compositions and methods of use for the purposes of illustration only. One skilled in the art will readily recognize from the following description that alternative embodiments of the present compositions and methods of use may be employed without departing from the principles of the invention described herein.

### DESCRIPTION

Unless otherwise defined, all technical and scientific terms used herein have the same meaning as is commonly understood by one of skill in the art to which this invention belongs and shall be understood to have the meanings described herein. All publications and patents referred to herein are incorporated by reference in their entirety.

Embodiments herein relate to improved pharmaceutical compositions comprising at least one cannabinoid component, or pharmaceutically acceptable salt thereof, and at least one motility agent component, or pharmaceutically acceptable salt thereof, and to methods of using same in the preparation of a medicament for treating, mitigating, or alleviating symptoms of IBS and IBS-related disorders in an individual.

Chronic Cannabis use can, in fact, increase an individual's risk of IBS. Counterintuitively, the present invention aims to provide a pharmaceutically active composition comprising at least one natural cannabinoid compound in combination with at least one motility agent, wherein the combination of cannabinoid and motility agent components may act synergistically to target IBS symptoms or IBS-related offset effects. Without being limited by theory, the present cannabinoid-based compositions may serve to either augment the positive or desired effects of the cannabinoids, and/or may serve to mitigate the negative or undesired effects of same, resulting in an improved Cannabis-based composition for treating, mitigating, or alleviating the symptoms of IBS or IBS-related disorder.

### Cannabinoid Constituents

According to embodiments, the present composition may comprise at least one first constituent derived from Cannabis plant material selected from Cannabis extract, Cannabis essential oil, or a combination thereof. In embodiments, the present composition may comprise at least one cannabinoid compound.

Herein, the term “cannabinoid” relates to the diverse class of chemical compounds acting on cannabinoid receptors, such compounds being in pure form, isolated from the Cannabis plant, and/or synthetically created. As known in the art, cannabinoids may generally comprise tetrahydrocannabinol (THC), the primary psychoactive compound in Cannabis as further described herein, cannabidiol (CBD), another major constituent of the plant as further described herein, and derivates or other structurally-related compounds. The term “cannabinoid”, as used herein, may also include synthetic cannabinoids, which encompass a variety of cannabinoids structurally related to, for example, THC, CBD, and the like. It is contemplated that the cannabinoids described herein may further include pharmaceutically acceptable salts, solvates, metabolites, metabolic precursors, isomer or derivatives of the cannabinoids.

The term “cannabinoid”, as used herein, may include phytocannabinoids, which are cannabinoids that occur naturally in the Cannabis plant (as well as other plants) and include over sixty unique compounds. As would be appreciated, phytocannabinoids are structurally similar to THC but with very different effects, some of which might be psychotropic.

The term “cannabinoid”, as used herein, may include terpenoids, a modified class of terpenes, which can be divided into several categories depending upon the number of carbon units (e.g. monoterpenes, sesquiterpenes, diterpenes, sesterpenes and triterpenes), and are a major constituent of the Cannabis sativa plant. Terpenoids are typically found to contain an integral number of C₅ units, chemically considered to be derived from the basic branched C₅ unit isoprene (2-methyl-1,3-butadiene), of which there are approximately 120 identified compounds.

Phytocannabinoids and terpenoids are known GRAs (“generally recognized as safe”) with the United States Food and Drug Administration, each having potentially unique impacts on an individual's gastrointestinal tract. Moreover, as would be appreciated, phytocannabinoids and terpenoids can each impact the microbiome directly, the microbiome being linked to gastrointestinal dysfunction. For example, cannabinoids can increase lipopolysaccharide levels that directly decrease gram negative bacteria in the microbiome. Without being limited by theory, the use of cannabinoids, phytocannabinoids and/or terpenoids to modulate the microbiome in order to directly modulate a disease state is a novel concept that could provide a further path to modulate the gastrointestinal system as outlined herein.

The term at least one “at least one cannabinoid” may relate to one cannabinoid or a combination of at least two cannabinoids, for example, any combination of two, three, four, five, six, ten or even more of any of the cannabinoids defined or described herein, whether naturally occurring cannabinoids, phytocannabinoids, chemically synthesized cannabinoids, or pharmaceutically acceptable salts thereof, solvates, metabolites, metabolic precursors, isomers or derivatives thereof.

The term “cannabinoid receptor” may relate to any one of the known subtypes of the class of cannabinoid receptors, including CB1 and/or CB2 receptors, that may be bound by the present cannabinoid-based compositions described herein. It follows that cannabinoids, as used herein, may include cannabinoid receptor (CB1 and/or CB2) agonists, partial agonists, or the like.

Cannabinoid receptors, located throughout the body, are part of the endocannabinoid system. Two of the primary cannabinoid receptors, known as CB₁ and CB₂, belong to the larger class of cell membrane receptors in the G protein-coupled receptor superfamily. G protein-coupled receptors are composed of seven transmembrane helices (spanning domains), and are activated by three major groups of ligands (i.e. endocannabinoids, cannabinoids, and synthetic cannabinoids). The CB₁ receptors, which are essentially expressed by the central and peripheral nervous systems, are known to cause a slowing-down of the peristalsis of the stomach and small intestine, and an inhibition of gastric secretion. Other anti-diarrheic functions of the cannabinoid receptors are also contemplated. Activation of the CB₂ receptors has been shown to modulate the immune system that might have direct impact on IBS symptoms especially if IBS is triggered by a microbiome dysfunction such as one seen in post infectious IBS. CB₁ receptors have also been seen to modulate intestinal barrier function that can protect the intestinal tract by modulating paracellular permeability.

According to embodiments, the present compositions may comprise at least one cannabinoid constituent. In some embodiments, as defined above, the at least one cannabinoid constituent may comprise tetrahydrocannabinol or “THC”, or more precisely its main isomer (−)-trans-Δ⁹-tetrahydrocannabinol, which is the main principal psychoactive cannabinoid of some stains of Cannabis. The THC may comprise the naturally occurring substance or synthetic versions of same, and includes, without limitation, pharmaceutically acceptable salts, solvates, metabolites, metabolic precursors, isomers, and derivatives thereof.

In some embodiments, as defined above, the at least one cannabinoid constituent may comprise cannabidiol or “CBD”, the major non-psychotropic cannabinoid in most strains of Cannabis. CBD, as used herein, may comprise the naturally occurring substance or synthetic versions of same, and includes, without limitation, pharmaceutically acceptable salts thereof, solvates, metabolites, metabolic precursors, isomers, and derivatives thereof.

In some embodiments, the at least one cannabinoid of the present compositions may be THC or a pharmaceutically acceptable salt thereof. In other embodiments, the at least one cannabinoid of the present compositions may be CBD or a pharmaceutically acceptable salt thereof. In yet other embodiments, the at least one cannabinoid of the present compositions may comprise at least two cannabinoids such as, without limitation, THC, CBD, or a combination thereof.

In some embodiments, the at least one cannabinoid of the present composition may comprise at least one phytocannabinoid.

In other embodiments, the at least one cannabinoid of the present composition may comprise at least one terpenoid. In some embodiments, the at least one terpenoid constituent of the present composition may comprise any suitable bioactive Cannabis terpenoid known in the art including, without limitation, limonene, myrcene, α-pinene, linalool, β-caryophyllene, caryophyllene oxide, nerolidol and phytol.

The number and amount of any at least one cannabinoid in the present composition may vary according to the intended use of the composition. For example, the present composition may comprise at least two cannabinoids, such as THC and CBD, in a 1:1 ratio, or other such pharmaceutically active ratio, for targeting IBS and IBS-related symptoms. It should be understood that, although the use of specific formulations consisting of THC/CBD may be described, other formulations of consisting of, without limitation, phytocannabinoids, terpenoids, and the like are contemplated. In typical embodiments, the at least one cannabinoid constituent may comprise at least 0.1-50% by weight (wt %) of the pharmaceutically active ingredient, and preferably at least 5-50% wt % of the pharmaceutically active ingredient. In certain embodiments, the at least one cannabinoid constituent may constitute 0.1-10 wt % of the active ingredient, 10-15 wt % of the active ingredient, 15-20 wt % of the active ingredient, 20-25 wt % of the active ingredient, 25-30 wt % of the active ingredient, 30-35 wt % of the active ingredient, 35-40 wt % of the active ingredient, 40-45 wt % of the active ingredient. In certain embodiments, the at least one cannabinoid constituent may constitute at least up to 5 wt %, at least 10 wt %, at least 15 wt %, at least 20 wt %, at least 30 wt %, at least 35 wt %, at least 40 wt %, at least 45 wt %, and may be at least 50 wt %. It is contemplated that the at least one cannabinoid may comprise at least 50-99.9 wt % of the active ingredient.

In some embodiments, the present compositions may comprise active, inactive, or slow acting, cannabinoids. It is contemplated that the inactive cannabinoids may convert or may be converted to active cannabinoids.

In some embodiments the present composition may be formulated to use a motility agent in combination with at least one cannabinoid receptor agonist that results in modulating disease state via the microbiome.

### Motility Agent Constituents

According to embodiments, the present composition may also include at least one second constituent comprising a motility agent, or a pharmaceutically acceptable salt thereof.

Herein, “motility agent” may relate to bioactive agents used in the management of intestinal motility disorders including, without limitation, prokinetic agents, gastrokinetic agents, propulsive agents, cholinergic agonists, opioid agonists or antagonists, antidiarrheals, antibiotics, or a combination thereof. A skilled person would appreciate that motility agents, as used herein, may be selected for their ability to react synergistically with the first at least one cannabinoid constituent of the composition in targeting IBS, IBS symptoms, or IBS-related offset effects including, for example, diarrhea, constipation, and/or pain. Although some or all of the motility agents described herein may show direct or indirect activity on the cannabinoid receptor, motility agents are excluded from the scope of cannabinoids, as such term is defined herein.

As above, according to embodiments, the present composition may comprise a mixture or blend of at least one cannabinoid constituent and at least one motility agent constituent whereby, in combination, a) both constituents improve or ameliorate symptoms of IBS in a synergistic fashion, and/or b) one of the first or second constituent may improve or ameliorate symptoms of IBS, while the other constituent offsets known negative side effects of the first.

Herein, examples of both first and second constituents for treating, mitigating or alleviating symptoms of IBS in a synergistic, additive or potentiating fashion (i.e. option a) above) may include, without limitation, a first cannabinoid constituent in combination with a second motility agent serving to alleviate diarrhea and/or pain. For example, the at least one motility agent may be selected from the group consisting of peripherally-restricted opioid receptor agonists such as meperidine (Demerol®), eluxadoline (Viberzi™, Truberzi™), loperamide (Imodium®), tapentadol (Nucynta™), diphenoxylate, oxymorphindole, or any other mu-, kappa-, or delta-opioid receptor agonist, and combinations thereof. The at least one motility agent may also be selected from the groups consisting of opioid receptor antagonists such as methylnaltrexone (Relistor™), naloxegol (Movantik™; Moventig™), naloxone (Narcan™), naldemedine (Symproic®). The at least one motility agent may comprise a combination or mixture of opioid receptor agonist and antagonist such as oxycodone and naloxone, or any other motility agent as might be appropriate so as act synergistically with the first cannabinoid constituent for treating, mitigating, or alleviating pain states.

In other embodiments, examples of first and second constituents might include a first cannabinoid constituent in combination with a second motility agent constituent comprising bile acid sequestrants, such as colestyramine or colestipol, commonly referred to as Questran® and Colestid®. In other embodiments, the second motility agent constituent may comprise other anti-diarrheal agents such as bismuth subsalicylate or subgallate (Pepto-Bismol®), attapulgite (Kaopectate®), or Crofelemer (Mytesi™).

In yet other embodiments, examples of first and second constituents might include a first cannabinoid constituent in combination with a second motility agent constituent comprising neurotransmitters within the enteric nervous system or which act upon the receptors for these neurotransmitters, i.e., neuromodulators, including acetylcholine (e.g. atropine, hyoscine, scopolamine, dicyclomine, belladonna, trihexyphenidyl, benztropine, bethanechol, anisotropine, clidinium bromide). Other neuromodulators including, without limitation, tachykinins (substance P, neurokinin A), nitric oxide, adenosine triphosphate, vasoactive intestinal peptide, neuropeptide Y, prostaglandins (misoprostol), serotonin or 5-hydroxytryptamine (5-HT) receptor agonists (prucalopride, renzapride, naronapride, cisapride, mosapride, tegaserod, trimebutine, ondansetron, granisetron, alosetron), and gut-specific calcium channel antagonists (pinaverium, mebeverine, otilonium) are also contemplated.

Herein, examples of the first or second constituent improving or ameliorating symptoms of IBS, while the other constituent offset potentially negative side effects of the first constituent (option b) above) may include, without limitation, at least one first cannabinoid constituent, which may help with abdominal pain but does not improve constipation, in combination with a second motility agent constituent, the second motility agent constituent comprising, without limitation, at least one anti-foaming or de-foaming agent, including simethicone or loperamide, commonly referred to as Gas-X®, osmotic or stimulant laxatives (e.g. PEG 3350, milk of magnesia, lactulose, aloe vera, mineral oil, senna, cascara, docusate), or stool softeners (docusate), and the like.

In other embodiments, examples of first and second constituents might include a first cannabinoid constituent in combination with a second motility agent constituent comprising enteric motor neuron modulators such as capsaicin, dietary fibers or other compounds operative to change the nature of the gastrointestinal tract by changing how nutrients or other chemicals are absorbed including, without limitation, soluble and insoluble fibers, psyllium, inulin (chicory inulin), vegetable gum (guar gum), beta-glucans, pectins and resistant starches. Microbiome modulators such as prebiotics, minimally absorbed antibiotics (rifaximin, neomycin), branched chain amino acids and prebiotics used to alleviate various gastrointestinal conditions by modulating the bacterial composition in the gut. It should be understood that other acceptable motility agents, or other ingredients, known in the art are contemplated.

The number and amount of any motility agent in the present composition may vary according to the intended use of the composition. For example, the present composition may comprise at least two motility agents in a 1:1 ratio, or other such pharmaceutically active ratio, for targeting IBS and IBS-related symptoms. In typical embodiments, the at least one motility agent constituent may comprise at least 50% by weight (wt %) of the pharmaceutically active ingredient, and preferably at least 0.1-50% wt % of the pharmaceutically active ingredient. In certain embodiments, the at least one motility agent constituent may constitute 0.1-10 wt % of the active ingredient, 10-15 wt % of the active ingredient, 15-20 wt % of the active ingredient, 20-25 wt % of the active ingredient, 25-30 wt % of the active ingredient, 30-35 wt % of the active ingredient, 35-40 wt % of the active ingredient, 40-45 wt % of the active ingredient. In certain embodiments, the at least one motility agent constituent may constitute at least up to 5 wt %, at least 10 wt %, at least 15 wt %, at least 20 wt %, at least 30 wt %, at least 35 wt %, at least 40 wt %, at least 45 wt %, and may be at least 50 wt %. It is contemplated that the at least one motility agent may comprise at least 50-99.9 wt % of the active ingredient.

It is an advantage that the present compositions aim to facilitate the co-administration of at least one first cannabinoid constituent and at least one second motility agent constituent for use in any need that may benefit from synergistic, additive, or potentiating therapeutic effect exerted by co-application of same, such as, but not limited to, some of the IBS and IBS-related offset effects that may be treated, mitigated, or alleviated by motility agent consumption when applied alone or cannabinoid consumption when applied alone.

Generally, according to embodiments, the present composition may comprise at least one cannabinoid constituent, the cannabinoid constituent comprising tetrahydrocannabinol (THC), cannabidiol (CBD), a phytocannabinoid, a terpenoid, and a combination thereof. The present composition may comprise a combination of THC and CBD.

In some embodiments, the at least one cannabinoid constituent may comprise at least one terpenoid, the at least one terpenoid selected from the group consisting of limonene, myrcene, α-pinene, linalool, β-caryophyllene, caryophyllene oxide, nerolidol and phytol, and a combination thereof.

In some embodiments, the present composition may also comprise at least one motility agent. The at least one motility agent may be at least one opioid receptor agonist, at least one opioid receptor antagonist, and a combination thereof.

In some embodiments, the at least one motility agent may comprise at least one opioid receptor agonist selected from the group consisting of a mu-, kappa-, and delta-opioid receptor agonist. In some embodiments, the at least one motility agent may be at least one opioid receptor agonist selected from the group consisting of meperidine, eluxadoline, loperamide, tapentadol. diphenoxylate, oxymorphindole, and a combination thereof.

In some embodiments, the at least one motility agent may comprise at least one opioid receptor antagonist selected from the group consisting of methylnaltrexone, naloxegol, naloxone, naldemedine.

In some embodiments, the present composition may also comprise at least one motility agent, the motility agent comprising a bile acid sequestrant. In some embodiments, the bile acid sequestrant may be selected from the group consisting of colestyramine and colestipol.

In some embodiments, the present composition may also comprise at least one motility agent, the motility agent comprising an anti-diarrheal agent. In some embodiments, the anti-diarrheal agent may be selected from the group consisting of bismuth subsalicylate, bismuth subgallate, attapulgite, crofelemer, and a combination thereof.

In some embodiments, the present composition may also comprise at least one motility agent, the motility agent comprising at least one enteric neuromodulator. In some embodiments, the at least one enteric neuromodulator is selected from the group consisting of atropine, hyoscine, scopolamine, dicyclomine, belladonna, trihexyphenidyl, benztropine, bethanechol, anisotropine, clinidium bromide, and combinations thereof.

In some embodiments, the present composition may also comprise at least one motility agent, the motility agent comprising at least one serotonin modulator. In some embodiments, the at least one serotonin modulator may be selected from the group consisting of prucalopride, renzapride, naronapride, cisapride, mosapride, tegaserod, trimebutine, ondansetron, granisetron, alosetron, and combinations thereof.

In some embodiments, the present composition may also comprise at least one motility agent, the motility agent comprising at least one gastrointestinal tract-specific calcium channel agonist. In some embodiments, the at least one gastrointestinal tract-specific calcium channel agonist may be selected from the group consisting of pinaverium, mebeverine, otilonium (e.g. otilonium bromide), and combinations thereof.

In some embodiments, the present composition may also comprise at least one motility agent, the motility agent comprising at least anti-foaming agent, defoaming agent, osmotic laxative, stimulant laxative, stool softeners, and combinations thereof. In some embodiments, the osmotic laxative, stimulant laxative, and stool softeners are selected from the group consisting of PEG 3350, milk of magnesia, lactulose, aloe vera, mineral oil, senna (e.g. senna glycoside), cascara, docusate, and combinations thereof.

In some embodiments, the present composition may also comprise at least one motility agent, the motility agent comprising at least one soluble fiber, at least one insoluble fiber, and a combination thereof. In some embodiments, the at least one motility agent comprising at least one soluble fiber and at least one insoluble fiber may be selected from psyllium, inulin, guar gum, beta-glucans, pectins, resistant starches, and a combination thereof.

In some embodiments, the present composition may also comprise at least one motility agent, the motility agent comprising at least one microbiome modulator. In some embodiments, the at least one microbiome modulator may be selected from the group consisting of at least one probiotic, rifaximin, metronidazole, neomycin, a branched chain amino acid, ciprofloxacin, L-ornithine, L-aspartate, at least one prebiotic, or a combination thereof.

### Methods

According to embodiments, the present invention comprises methods for preparing the present novel cannabinoid-based compositions, or pharmaceutically acceptable salts thereof. In other embodiments, the present invention comprises the use of the present novel cannabinoid-based compositions, or pharmaceutically acceptable salts thereof, for the manufacture of a medicament. Such use of the present compositions, or salts thereof, may comprise administering a pharmaceutically effective amount of at least one novel cannabinoid-based composition to an individual. It should be understood that the foregoing methods of preparation, of use, and of manufacture of a medicament of the present novel cannabinoid-based compositions may be for target IBS and IBS-related symptoms in an individual suffering from same. More specifically, it is contemplated that the present methods of preparation, of use, and of manufacture of a medicament of the present novel cannabinoid-based compositions may be for the treatment, mitigation, or alleviation of IBS and IBS-related symptoms in an individual suffering from same. Other uses of the present novel cannabinoid-based compositions as might be known in the art are also contemplated.

Herein, the term “pharmaceutical composition” may relate to a preparation containing at least one cannabinoid and at least one motility agent, as described herein, or pharmaceutically acceptable salts thereof, with other chemical components including, but not limited to, pharmaceutically suitable carriers, excipients (e.g. non-active substances for facilitating processes and administration of the active ingredients), lubricants, buffering agents, and the like. The purpose of the present pharmaceutical composition is to facilitate administration of the active agents, in combination, to an individual, and in particular a human. The term “active ingredient” refers to a compound, which is accountable for the desired biological effect. The term “pharmaceutically acceptable carrier” refers to an approved carrier or diluent that does not cause significant irritation to an organism and does not abrogate the biological activity and properties of a possible active agent.

Herein the terms “treating”, “mitigating”, or “alleviating” may include:
a. preventing or delaying the appearance of clinical symptoms developing in an individual that may be afflicted with or predisposed to the symptoms, state, disorder, or condition, but does not yet experience or display clinical or subclinical symptoms of the state, disorder or condition;
b. inhibiting the state, disorder or condition, i.e., arresting or reducing the development of the disease or at least one clinical or subclinical symptom thereof; or
c. relieving the disease, i.e., causing regression of the state, disorder or condition or at least one of its clinical or subclinical symptoms.

Herein, a “therapeutically effective amount”, “pharmaceutically bioactive amount” or the like means the amount of the present compositions that, when used to prepare a medicament or when administered to an individual for treating, mitigating, or alleviating symptoms, a state, disorder or condition, is sufficient to affect such treatment, i.e. to provide a therapeutic effect. The “therapeutically effective amount” will vary depending on the particular composition and its constituents, the disease and its severity, and the age, weight, physical condition and responsiveness of the individual to be treated. It is contemplated that the present therapeutic effect is selected from a synergistic effect, an additive effect, a potentiating effect, and any combination thereof. For example, methods herein comprise providing the present composition to an individual in need thereof a pharmaceutically active amount of the compositions, such methods resulting in a synergistic effect while treating, mitigating, or alleviating the symptoms of IBS or IBS-related disorders. It is further contemplated that the present therapeutic effect may be at least 100% or higher than the therapeutic effect of the at least one cannabinoid or the at least one motility agent administered separately in a similar amount.

According to embodiments, the present methods may comprise treating, mitigating, or alleviating the symptoms of IBS or IBS-related offset effect in an individual. In some embodiments, methods of treating, mitigating, or alleviating the symptoms may comprise administering, to the individual, a therapeutically effective amount of at least one pharmaceutically active composition comprising at least one cannabinoid and at least one synergistic motility agent, as defined herein.

In some embodiments, the present methods may comprise treating, mitigating, or alleviating the symptoms of IBS or IBS-related offset effects by administering the present pharmaceutically active compositions to an individual suffering from same. The present methods may comprise administering the present pharmaceutically active composition pro re nata (PRN, “as needed”, or “when necessary”).

In other embodiments, the present methods may comprise the use of the present pharmaceutically active compositions for treating, mitigating, or alleviating the symptoms of IBS or IBS-related offset effects in an individual suffering from same. In some embodiments, the use of the present pharmaceutically active composition may comprise using the present pharmaceutically active composition in preparing a medicament for treating, mitigating, or alleviating the symptoms of IBS or IBS-offset effects in an individual.

Generally, according to embodiments, the methods provided herein comprise administration of a composition as described herein. In some embodiments, the present methods may comprise formulating the compositions for administration to an individual suffering from irritable bowel syndrome or irritable bowel syndrome-related symptoms. In some embodiments, the present methods may comprise formulating the present compositions for administration to an individual for the treatment, mitigation, or alleviation of irritable bowel syndrome or irritable bowel syndrome-related symptoms. In some embodiments, the present methods may comprise formulating a pharmaceutically active amount of the composition for administration to an individual. In some embodiments, the present methods may comprise formulating the present compositions for use in the preparation of a medicament used to treat, mitigate, or alleviate the symptoms of irritable bowel syndrome or irritable bowel syndrome-related symptoms in an individual.

In some embodiments, methods are provided for treating, mitigating, or alleviating the symptoms of irritable bowel syndrome or irritable bowel syndrome-related symptoms. In some embodiments, the present methods comprise administering a pharmaceutically active amount of the present compositions to an individual suffering from irritable bowel syndrome or irritable bowel syndrome-related symptoms.

Herein, it is contemplated that the present pharmaceutically active compositions may be provided, administered, and used in any manner suitable in the art. Specifically, it is contemplated that the dosage and delivery of the present compositions may be formulated in accordance with any suitable procedures known in the art.

According to embodiments, the present compositions may be adapted for use daily, weekly, or monthly. In some embodiments, the present pharmaceutical composition may be administered at least once a day, at least more than once a day, once a week, at least more than once a week, monthly or at least more than once a month.

According to embodiments, the present pharmaceutically active composition may be in any form appropriate for human medicine including a liquid, an oil, an emulsion, a gel, a colloid, an aerosol, or a solid. The present compositions may be formulated for administration by any appropriate route of administration known in the art including, without limitation, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some embodiments, it can be desirable to introduce the present compositions into the central nervous system or gastrointestinal tract by any suitable route, including intraventricular, intrathecal, and epidural injection, and enema. The mode of administration may be at the discretion of the individual and/or a medical professional. In some instances, administration of the present compositions may result in the release of the present compositions into the bloodstream. In some embodiments, the present compositions may be delivered in a controlled-release system or sustained-release format.

According to embodiments, the present compositions may be adapted for oral administration to an individual. Compositions for oral administration may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin, and flavoring, colouring or preserving agents, to provide a pharmaceutically palatable preparation. Moreover, where in tablet or pill form, the compositions can be coated to delay disintegration and absorption (e.g. in the gastrointestinal tract) thereby providing a controlled or sustained action over an extended period of time. In this regard, a time-delay material of pharmaceutical grade may be used.

As would be appreciated by a person skilled in the art, controlled- or sustained-release compositions can serve to initially release an amount of the at least one cannabinoid agonist for achieving the desired therapeutic effect, and gradually and continually release other amounts of the at least one synergistic agent (or vice versa). The amount of the at least one cannabinoid and/or synergistic agent may be an amount that is effective in the treatment of IBS and IBS-related symptoms in humans. Various techniques, including in vitro or in vivo assays may optionally be performed to assist in identifying optimal dosage ranges, whereby the optimal dose may be dependent upon the mode of administration.

By way of example, the present compositions may comprise at least one first cannabinoid constituent comprising a combination of THC and CBD, and preferably in a 1:1 ratio, and at least one second motility agent constituent, and preferably loperamide, wherein both first and second constituent act to reduce motility in the gastrointestinal tract. However, without being limited to theory, the reduction of motility by such a composition may be mediated by two very different pathways (mu opioid receptors vs. CB receptors), the synergistic effect thus not being purely additive and resulting in the combination of constituents being non obvious. For instance, while common side effects of loperamide include nausea, cramping, and constipation, the foregoing example compositions activation of CB receptors would serve to alleviate the very same symptoms. Furthermore, CB1 receptor agonists have shown to modulate enteric neural metabolic activity allowing for a prolonged effect, therefore a time effect can be staggered, tuned, and optimized for each formulation of the present composition.

According to embodiments, the present first and second constituents may be formulated with any convenient pharmaceutically acceptable diluents, carriers, or excipients to produce the pharmaceutically active composition. Examples, without limitation, of excipients can include calcium carbonate, calcium phosphate, various sugars and types of starch, cellulose, derivatives, gelatin, vegetable oils, polyethylene glycols, and any other excipients as may be known in the art to be suitable. The present first and second constituents may be mixed with one or more fillers or extenders, binders, humectants, disintegrating agents, solution retarding agents, absorption accelerators, wetting agents, absorbents, lubricants, colouring agents, or combinations thereof. The choice of diluents, carriers, excipients, or the like, may depend on the desired dosage form, which may in turn be dependent on the mode of administration. Such dosage forms may be prepared in accordance with standard principles of pharmaceutical formulation, known to those skilled in the art.

In some embodiments, the at least one first and second constituents of the present compositions may be administered or consumed together at the same time, or, alternatively, they may be administered or consumed separately (e.g. according to a predetermined regimen). For example, co-administration of the at least one first and second constituents of the present compositions may be carried out by providing an individual in need thereof the present compositions formulated as a single unit dose form. Additionally, co-administration of the at least one first and second constituents of the present composition may be carried out by providing an individual in need thereof two or more units of dosage form, at least one of which comprises the at least one first cannabinoid constituent and at least one of which comprises the at least one second motility agent constituent.

Although a few embodiments have been shown and described, it will be appreciated by those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and the described portions thereof.

We claim:

1) A pharmaceutically active composition comprising:
at least one cannabinoid, and
at least one motility agent.

2) The composition of claim 1, wherein the at least one cannabinoid is selected from the group consisting of tetrahydrocannabinol (THC), cannabidiol (CBD), a phytocannabinoid, a terpenoid, and a combination thereof.

3) The composition of claim 2, wherein the at least one cannabinoid comprises a combination of THC and CBD.

4) The composition of claim 3, wherein the at least one cannabinoid comprising THC and CBD in a 1:1 ratio.

5) The composition of claim 2, wherein the at least one terpenoid is selected from the group consisting of limonene, myrcene, α-pinene, linalool, β-caryophyllene, caryophyllene oxide, nerolidol and phytol, and a combination thereof.

6) The composition of claim 1, wherein the at least one motility agent comprises at least one opioid receptor agonist, at least one opioid receptor antagonist, and a combination thereof.

7) The composition of claim 6, wherein the at least one opioid receptor agonist is a mu-, kappa-, and delta-opioid receptor agonist.

8) The composition of claim 6, wherein the at least one opioid receptor agonist is selected from the group consisting of meperidine, eluxadoline, loperamide, tapentadol. diphenoxylate, oxymorphindole, and a combination thereof.

9) The composition of claim 1, wherein the at least one cannabinoid comprises a combination of THC and CBD and the at least one motility agent comprises loperamide.

10) The composition of claim 6, wherein the at least one opioid receptor antagonist is selected from the group consisting of methylnaltrexone, naloxegol, naloxone, naldemedine.

11) The composition of claim 1, wherein the at least one motility agent comprises a bile acid sequestrant selected from the group consisting of colestyramine and colestipol.

12) The composition of claim 1, wherein the at least one motility agent comprises an anti-diarrheal agent selected from the group consisting of bismuth subsalicylate, bismuth subgallate, attapulgite, crofelemer, and a combination thereof.

13) The composition of claim 1, wherein the at least one motility agent comprises at least one enteric neuromodulator selected from the group consisting of atropine, hyoscine, scopolamine, dicyclomine, belladonna, trihexyphenidyl, benztropine, bethanechol, anisotropine, clinidium bromide, and combinations thereof.

14) The composition of claim 1, wherein the at least one motility agent comprises at least one serotonin neuromodulators selected from the group consisting of prucalopride, renzapride, naronapride, cisapride, mosapride, tegaserod, trimebutine, ondansetron, granisetron, alosetron, and combinations thereof.

15) The composition of claim 1, wherein the at least one motility agent comprises at least one gastrointestinal tract-specific calcium channel agonist selected from the group consisting of pinaverium, mebeverine, otilonium, and combinations thereof.

16) The composition of claim 1, wherein the at least one motility agent comprises at least one anti-foaming agent, defoaming agent, osmotic laxative, stimulant laxative, stool softeners, and combinations thereof.

17) The composition of claim 16, wherein the osmotic laxative, stimulant laxative, and stool softeners are selected from the group consisting of PEG 3350, milk of magnesia, lactulose, aloe vera, mineral oil, senna, cascara, docusate, and combinations thereof.

18) The composition of claim 1, wherein the at least one motility agent comprises at least one soluble and insoluble fibers selected from the group consisting of psyllium, inulin, guar gum, beta-glucans, pectins, resistant starches, and a combination thereof.

19) The composition of claim 1, wherein the at least one motility agent comprises at least one microbiome modulator selected from the group consisting of at least one probiotic, rifaximin, metronidazole, neomycin, a branched chain amino acid, ciprofloxacin, L-ornithine, L-aspartate, at least one prebiotic, or a combination thereof.

20) The composition of any one of claims 1-19, wherein the composition is formulated for administration to an individual suffering from irritable bowel syndrome or irritable bowel syndrome-related symptoms.

21) The composition of claim 20, wherein the composition is formulated for administration to an individual for the treatment, mitigation, or alleviation of irritable bowel syndrome or irritable bowel syndrome-related symptoms.

22) Use of the composition in any one of claims 1-19 comprising administering a pharmaceutically active amount of the composition to an individual for treating, mitigating, or alleviating the symptoms of irritable bowel syndrome or irritable bowel syndrome-related symptoms in the individual.

23) Use of the composition in any one of the claims 1-19 for the preparation of a medicament for treating, mitigating, or alleviating the symptoms of irritable bowel syndrome or irritable bowel syndrome-related symptoms in an individual.

24) A method of treating, mitigating, or alleviating the symptoms of irritable bowel syndrome in an individual comprising administering a therapeutically effective amount of a pharmaceutically active composition comprising at least one cannabinoid and at least one motility agent to the individual.

25) The method of claim 24, wherein the at least one cannabinoid comprises at least tetrahydrocannabinol (THC), cannabidiol (CBD), or a combination thereof.
